(19) United States
(12) Patent Application Publication
(10) Pub. No.: US 2025/0369964 A1
(43) Pub. Date: Dec. 4, 2025

(54) TEST KIT AND METHOD OF DESIGNING TEST KIT

(71) Applicant: CANON MEDICAL SYSTEMS CORPORATION, Otawara-shi (JP)

(72) Inventors: Keisuke SUGAHARA, Nasushiobara (JP); Shunsuke FUJIWARA, Utsunomiya (JP)

(73) Assignee: CANON MEDICAL SYSTEMS CORPORATION, Otawara-shi (JP)

(21) Appl. No.: 19/218,871

(22) Filed: May 27, 2025

(30) Foreign Application Priority Data
May 28, 2024 (JP) 2024-086343

(51) Int. Cl. G01N 33/543; G01N 21/77; G01N 21/78; G01N 33/53

(52) U.S. Cl. CPC G01N 33/54388; G01N 21/78; G01N 33/5308; G01N 2021/7759; G01N 2333/922

(57) ABSTRACT

A test kit for detecting a target nucleic acid sequence in a specimen according to an embodiment includes a lateral flow assay (LFA) test paper. The LFA test paper tests a reaction solution obtained by adding the specimen to a reaction solution containing a Cas enzyme and a reporter molecule precursor and subjecting the reaction solution to a CRISPR-Cas reaction. The LFA test paper includes a labeling area containing labeled antibodies, a first detection line to which capture molecules for capturing the reporter molecule precursor are fixed, and a second detection line to which capture antibodies for capturing the labeled antibodies are fixed. The amount A of the labeled antibodies and the amount S of the capture molecules on the LFA test paper satisfy conditions of A≥10⁻¹² mol and 1≤S/A.

### CROSS-REFERENCE TO RELATED APPLICATION

The present application claims priority based on Japanese Patent Application No. 2024-086343 filed May 28, 2024, the content of which is incorporated herein by reference.

### FIELD

Embodiments disclosed in the present specification and drawings relate to a test kit and a method of designing the test kit.

### BACKGROUND

A nucleic acid detection method (CRISPR-Cas LFA test) that combines CRISPR-Cas, which is a genome editing technique, and lateral flow assay (LFA), which is a test method based on an antigen-antibody reaction, has been known. In this CRISPR-Cas LFA test, for example, a specimen containing DNA or RNA extracted from a subject is added to a reaction solution containing a Cas enzyme and a reporter molecule precursor and subjected to a CRISPR-Cas reaction to generate a sample containing a reporter molecule precursor and/or a reporter molecule, and the generated sample is dropped onto an LFA test paper to perform a test to determine presence or absence (negative/positive) of the target nucleic acid in the specimen. For example, the reporter molecule precursor has a structure in which a first antigen and a second antigen are modified at both ends of a single-stranded DNA. Further, the LFA test paper is provided with a labeling area where a labeled antibody of the first antigen is placed, a first detection line to which a capture molecule that captures the second antigen is fixed, and a second detection line to which a capture antibody of the labeled antibody is placed.

### BRIEF DESCRIPTION OF THE DRAWINGS

FIG. 1 is a diagram showing an example of a test kit according to an embodiment.

FIG. 2 is a diagram showing a CRISPR-Cas reaction process according to the embodiment.

FIG. 3A is a diagram showing a target nucleic acid detection flow (detection principle) under negative conditions according to the embodiment.

FIG. 3B is a diagram showing a target nucleic acid detection flow (detection principle) under negative conditions according to the embodiment.

FIG. 4A is a diagram showing a target nucleic acid detection flow (detection principle) under positive conditions according to the embodiment.

FIG. 4B is a diagram showing a target nucleic acid detection flow (detection principle) under positive conditions according to the embodiment.

FIG. 5A is a graph showing change in the amount of labeled antibodies on each detection line relative to the amount Rp of reporter molecule precursor introduced under negative conditions for an LFA test paper in the related art.

FIG. 5B is a graph showing change in the amount of labeled antibodies on each detection line relative to the amount Rp of reporter molecule precursor introduced under negative conditions for the LFA test paper in the related art.

FIG. 6A is a graph showing change in the amount of labeled antibodies on each detection line relative to the amount Rp of reporter molecule precursor introduced under negative conditions for an LFA test paper of an embodiment.

FIG. 6B is graph showing change in the amount of labeled antibodies on each detection line relative to the amount Rp of reporter molecule precursor introduced under negative conditions for the LFA test paper of the embodiment.

FIG. 7 is a diagram showing a relationship between an allowable range of the amount Rp of reporter molecule precursor introduced (Rp allowable range) and the ratio S/A of the amount S of capture molecules to the amount A of labeled antibodies on a first detection line according to an embodiment when the ratio S/A is changed.

FIG. 8A is a diagram showing change in color intensity with respect to the amount of reporter molecule precursor on the first detection line according to an embodiment.

FIG. 8B is a diagram showing change in color intensity with respect to the amount of reporter molecule precursor on the second detection line according to the embodiment.

FIG. 9A is a graph showing a relationship between the amount of labeled antibodies and color intensity according to the embodiment.

FIG. 9B is an enlarged view of a portion of the graph shown in FIG. 9A.

FIG. 10A is a graph showing change in specificity when the ratio S/A of the amount S of capture molecules to the amount A of labeled antibodies on the first detection line according to the embodiment is changed.

FIG. 10B is a graph showing change in specificity with respect to variation (standard deviation) in the amount Rp of reporter molecule precursor introduced according to the embodiment.

FIG. 11 is a graph showing change in sensitivity with respect to variation (standard deviation) in the amount Rp of reporter molecule precursor introduced according to the embodiment.

### DETAILED DESCRIPTION

Hereinafter, a test kit and a method of designing the test kit according to an embodiment will be described with reference to the drawings.

In CRISPR-Cas LFA test kits in the related art, it was not uncommon for a second detection line to turn colored, resulting in a false positive result, even under negative conditions. As will be described below, the inventor of the present invention has found that one of the causes of this false positive result is the tolerance to variations in the amount of reporter molecule precursor introduced.

A test kit for detecting a target nucleic acid sequence in a specimen according to an embodiment includes a lateral flow assay (LFA) test paper. The LFA test paper is used to test a reaction solution obtained by adding a specimen to a reaction solution containing a Cas enzyme and a reporter molecule precursor and subjecting the reaction solution to a CRISPR-Cas reaction. The LFA test paper includes a labeling area including labeled antibodies, a first detection line to which capture molecules that capture the reporter molecule precursor are fixed, and a second detection line to which capture antibodies that capture the labeled antibodies are fixed. The amount A of the labeled antibodies and the amount S of the capture molecules on the LFA test paper satisfy the conditions of A≥10⁻¹² mol and 1≤S/A. This makes it possible to carry out a robust test against variations in the amount of reporter molecule precursor introduced.

### Configuration of Test Kit

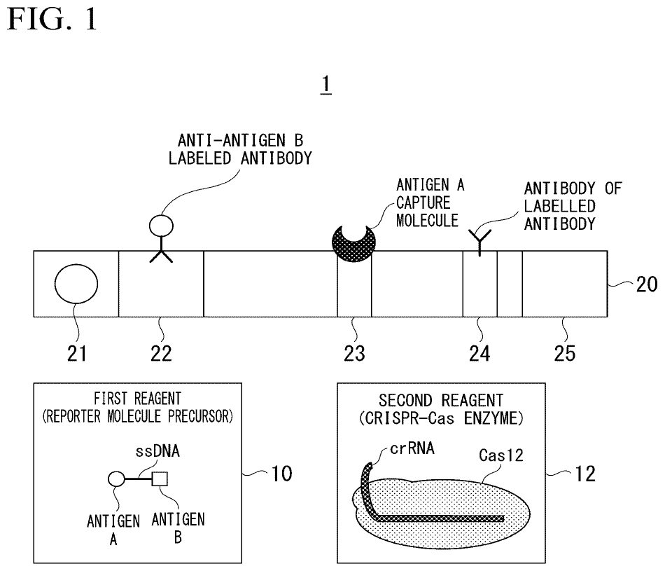

First, a configuration of a test kit according to an embodiment will be described. FIG. 1 is a diagram showing an example of a test kit 1 according to an embodiment. The test kit 1 is used to perform a CRISPR-Cas LFA test. The test kit 1 detects a nucleic acid (hereinafter referred to as a “target nucleic acid”) that is a target (subject) of detection. The target nucleic acid includes, for example, deoxyribonucleic acid (DNA) and ribonucleic acid (RNA). The target nucleic acid may be single-stranded or double-stranded. The target nucleic acid is, for example, derived from a microorganism having a pathogen. The microorganism includes, for example, a virus, a bacteria, and the like.

The test kit 1 includes, for example, a first reagent 10, a second reagent 12, and an LFA test paper 20 (device). The first reagent 10 includes a reporter molecule precursor. The reporter molecule precursor includes a nucleic acid (DNA or RNA) that is a substrate for the non-specific nucleic acid cleavage activity of the CRISPR-Cas enzyme. Both ends of the nucleic acid in the reporter molecule precursor are modified with a molecule (antigen B) for binding to a labeled antibody and a molecule (antigen A) for binding to a capture molecule of the first detection line 23. The antigen B is, for example, 5-carboxyfluorescein (FAM). The antigen A is, for example, biotin. The nucleic acid sequence may be any sequence. The second reagent 12 contains a CRISPR-Cas enzyme. The CRISPR-Cas enzyme is a complex of a Cas protein (Cas12a, Cas12b, Cas12c, etc.) and RNA (crRNA). A specimen is added to a reaction solution containing the first reagent 10 (reporter molecule precursor) and the second reagent 12 (CRISPR-Cas enzyme) to cause a CRISPR-Cas reaction. A test using the LFA test paper 20 is performed on the CRISPR-Cas reaction solution after such a CRISPR-Cas reaction.

The main material of the LFA test paper 20 is, for example, a material used in immunochromatography tests in the related art. The main material of the LFA test paper 20 is, for example, nitrocellulose and the like. The LFA test paper 20 can be, for example, 60 mm long and 3 mm wide as an example of size. The LFA test paper 20 includes, for example, a sample pad 21, a labeling area 22 (labeled antibody pad (conjugation pad)), a first detection line 23, a second detection line 24, and an absorbent pad 25. The sample pad 21 onto which the CRISPR-Cas reaction solution is dropped is located at the most upstream position, followed by the labeling area 22, the first detection line 23, the second detection line 24, and the absorbent pad 25 in this order.

The sample pad 21 sends the dropped CRISPR-Cas reaction solution to the labeling area 22. As in immunochromatographic tests in the related art, the sample pad 21 is made of, for example, a cellulose fiber filter.

The labeling area 22 contains an anti-antigen B labeled antibody that binds to the antigen B of the reporter molecule precursor. The anti-antigen B labeled antibody contains an antibody that binds to the reporter molecule precursor or reporter molecule, and a labeled molecule that can show color on the LFA test paper surface. The anti-antigen B labeled antibody is, for example, an anti-FAM antibody labeled with gold nanoparticles. The labeling area 22 allows the reporter molecule precursor or the antigen B of the reporter molecule flowing from the sample pad 21 to form a complex with the anti-antigen B labeled antibody in the labeling area 22 while flowing downstream. The labeling area 22 is made of, for example, glass fiber which is used in immunochromatographic tests in the related art, or the like.

The first detection line 23 is an area where a molecule that captures the reporter molecule precursor is fixed. The first detection line 23 shows color when the captured reporter molecule precursor binds to a labeled antibody (anti-antigen B labeled antibody). The first detection line 23 is provided to determine a negative condition in a test for a target nucleic acid. An antigen A capture molecule that binds to the antigen A, which is a modified molecule of the reporter molecule precursor, is fixed to the first detection line 23. The antigen A capture molecule is, for example, streptavidin. When the antigen A of the reporter molecule precursor bound to the anti-antigen B labeled antibody binds to the antigen A capture molecule, the anti-antigen B labeled antibody remains on the first detection line 23, and coloration of the first detection line 23 occurs.

The second detection line 24 is an area where a molecule that binds to the labeled antibody (anti-antigen B labeled antibody) flowing from the first detection line 23 is fixed. The second detection line 24 shows color by the captured labeled antibody. The second detection line 24 is provided to determine a positive condition in a test for a target nucleic acid. An antibody (capture antibody) of a labeled antibody, which is a molecule that binds to the anti-antigen B labeled antibody, is fixed to the second detection line 24. The antibody of the labeled antibody is, for example, rabbit IgG. When the anti-antigen B labeled antibody bound to the reporter molecule (antigen B) binds to the antibody of the labeled antibody, the anti-antigen B labeled antibody remains on the second detection line 24, and coloration of the second detection line 24 occurs.

The absorbent pad 25 is provided furthest downstream on the LFA test paper 20 and absorbs the flowing CRISPR-Cas reaction solution. The absorbent pad 25 is made of, for example, cellulose which is used in immunochromatography tests in the related art, or the like.

### Testing Method

Next, each process in a testing method using the test kit 1 will be described. This testing method includes a CRISPR-Cas reaction process and a target nucleic acid detection process (LFA process).

### CRISPR-Cas Reaction Process

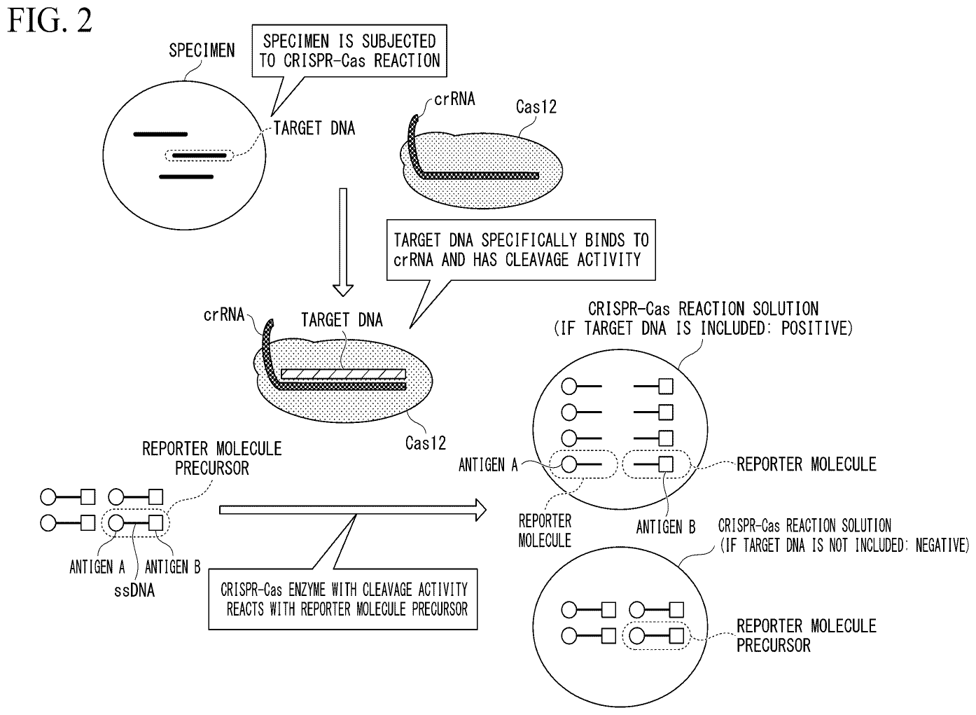

First, the CRISPR-Cas reaction process will be described. FIG. 2 is a diagram showing the CRISPR-Cas reaction process according to an embodiment. For example, in this process, a specimen is added to a reaction solution containing a CRISPR-Cas enzyme and a reporter molecule precursor to cause a CRISPR-Cas reaction. The CRISPR-Cas enzyme recognizes a target nucleic acid (DNA or RNA) sequence by crRNA and has the activity of non-specifically cleaving the surrounding nucleic acid (DNA or RNA). In the CRISPR-Cas reaction, when a nucleic acid having a target sequence is present in the specimen, the CRISPR-Cas enzyme is activated and cleaves the nucleic acid of the reporter molecule precursor to convert it into a reporter molecule. As an example of CRISPR-Cas reaction conditions, 20 μL of reaction solution is prepared in a PCR tube, and the PCR tube is reacted in a thermostatic bath at 37° C. for 30 to 60 minutes.

When the specimen contains DNA to be detected (hereinafter referred to as “target DNA”) (i.e., a “positive” case), this target DNA specifically binds to the crRNA of the CRISPR-Cas enzyme. When bound to the target DNA, the CRISPR-Cas enzyme has cleavage activity for nucleic acid of any sequence. Here, a cleavage reaction of ssDNA of the reporter molecule precursor occurs by the activated CRISPR-Cas enzyme. As a result, a reporter molecule (antigen A reporter molecule, antigen B reporter molecule) in which the ssDNA of the reporter molecule precursor has been cleaved is generated.

On the other hand, when the specimen does not contain the target DNA to be detected (i.e., a “negative” case), the target DNA does not bind to the crRNA, and thus the CRISPR-Cas enzyme does not have cleavage activity. Here, no cleavage reaction of ssDNA of reporter molecule precursor occurs because the CRISPR-Cas enzyme does not have the cleavage activity.

### Target Nucleic Acid Detection Process (LFA Process)

Next, the target nucleic acid detection process (LFA process) will be described. In this process, the CRISPR-Cas reaction solution (hereinafter also referred to as a “sample”) after the CRISPR-Cas reaction is mixed with a buffer to adjust the pH, viscosity, and the like. As an example, 100 μL of buffer is added to 10 μL of sample. For example, a Tris Buffered Saline-based buffer (HybriDetect Assay Buffer, #MGCB, Milenia Biotec GmbH) is used as the buffer. Next, the sample mixed with the buffer is poured into the LFA test paper 20. For example, 10 μL of sample is dropped onto the sample pad 21 and allowed to flow through the LFA test paper 20 for 5 minutes, and coloration of each detection line is determined.

Alternatively, the sample mixed with the buffer may be placed in a microtube, and the LFA test paper 20 may be immersed in the sample to introduce the sample and then removed after 5 minutes to perform a test. The presence or absence (positive/negative) of the target nucleic acid sequence in the specimen can be determined from the coloration state of each detection line of the LFA test paper 20 after the sample has flowed. For example, if the first detection line 23 is colored red and the second detection line 24 is not colored by visual determination, it is determined to be negative, and if the second detection line 24 is colored regardless of the presence or absence of coloration of the first detection line 23, it is determined to be positive. Alternatively, a measuring device such as an immunochromatography reader may be used to determine coloration states based on whether a measurement result is equal to or greater than a threshold value, and negative/positive determination may be performed.

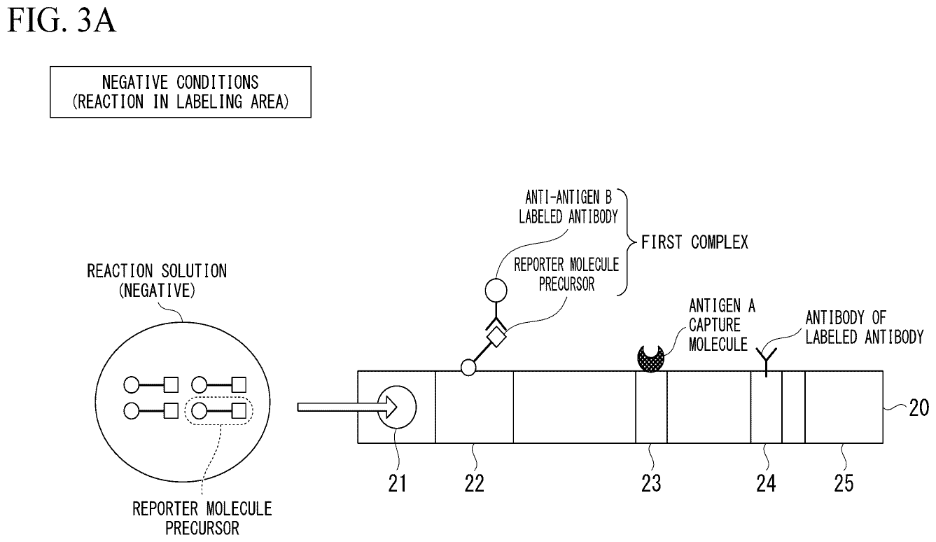

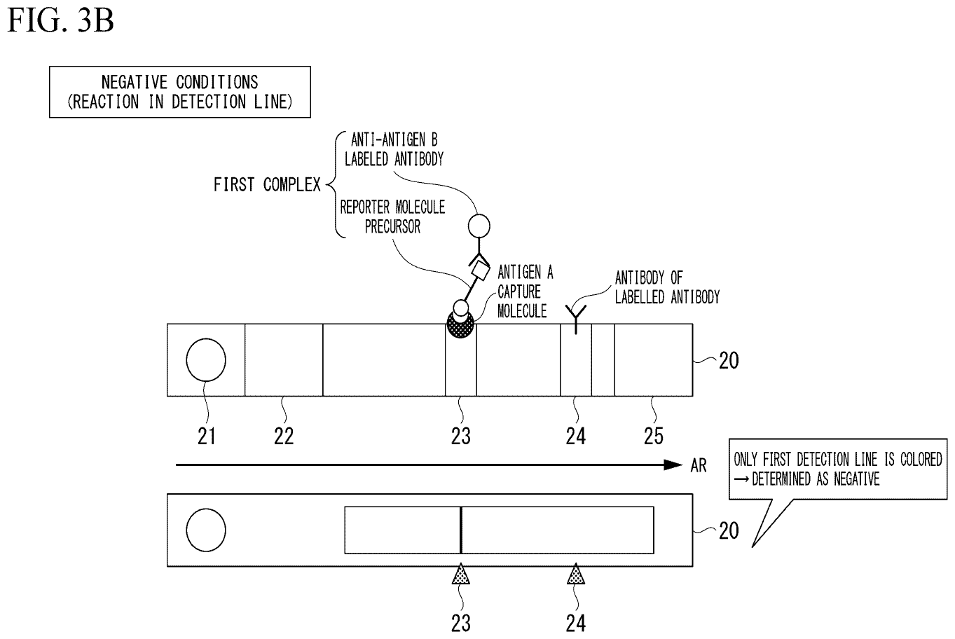

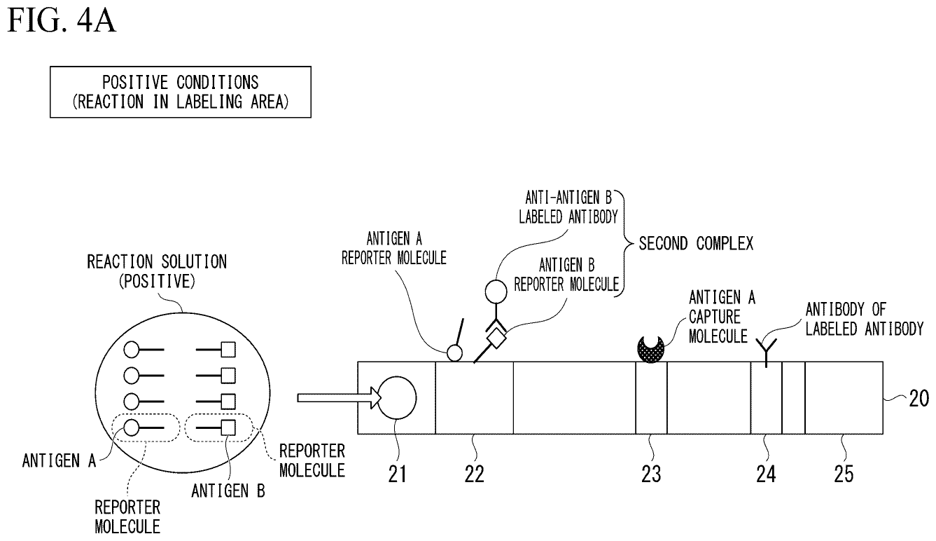

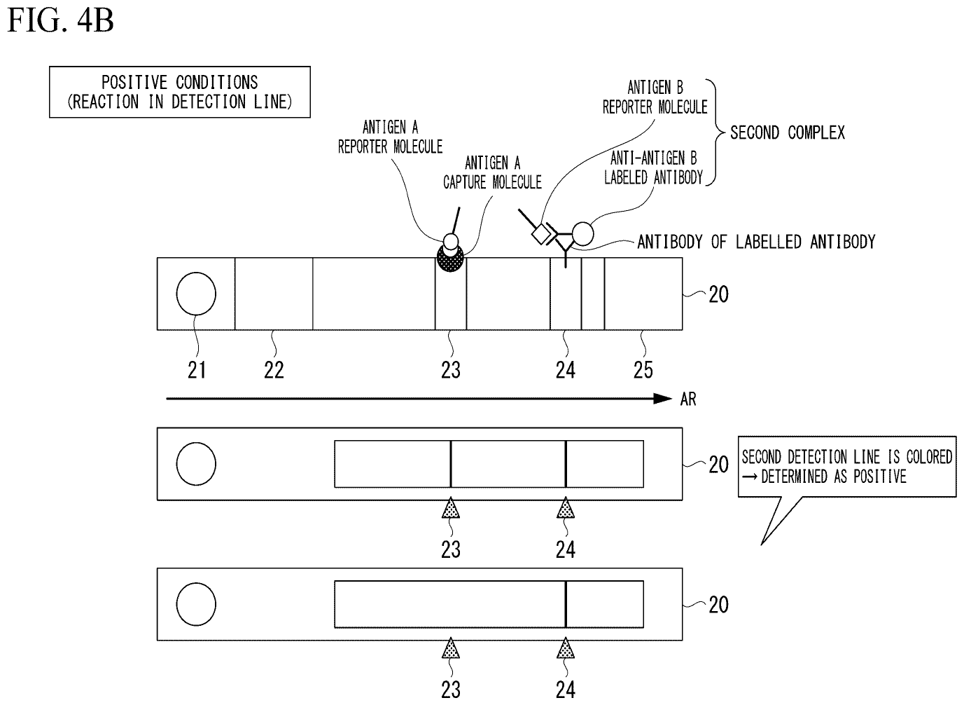

FIG. 3A and FIG. 3B are diagrams showing a target nucleic acid detection principle (detection principle) under negative conditions according to the embodiment. On the other hand, FIG. 4A and FIG. 4B are diagrams showing a target nucleic acid detection principle (detection principle) under positive conditions according to the embodiment.

### Negative Conditions

As shown in FIG. 3A, a sample of the CRISPR-Cas reaction solution is dropped onto the sample pad 21 of the LFA test paper 20. Under negative conditions, this sample contains a reporter molecule precursor but does not contain a reporter molecule. The sample dropped onto the sample pad 21 flows into the labeling area 22, and a complex (hereinafter referred to as a “first complex”) is generated by binding the anti-antigen B labeled antibody that placed in the labeling area 22 and the antigen B of the reporter molecule precursor contained in the sample.

Next, as shown in FIG. 3B, the generated first complex flows in the direction of the arrow AR in the LFA test paper 20 by capillary force, and reaches the first detection line 23. The first complex (antigen A at one end of the reporter molecule precursor) that has reached the first detection line 23 binds to the antigen A capture molecule fixed to the first detection line 23. An increase in the amount of the first complex bound to such antigen A capture molecule causes coloration of the detection line on the first detection line 23. On the other hand, no coloration occurs in the detection line on the second detection line 24. By checking the coloration of only the detection line on the first detection line 23, the inspector can confirm that the target nucleic acid has not been detected and that the test result is negative.

### Positive Conditions

As shown in FIG. 4A, a sample of the CRISPR-Cas reaction solution is dropped onto the sample pad 21 of the LFA test paper 20. Under positive conditions, the sample contains reporter molecules (antigen A reporter molecule, antigen B reporter molecule). Depending on the amount of reporter molecule precursor, the sample may contain an unreacted reporter molecule precursor. The sample dropped onto the sample pad 21 flows into the labeling area 22, and a complex (hereinafter referred to as a “second complex”) is generated by binding the anti-antigen B labeled antibody that placed in the labeling area 22 and the antigen B reporter molecule contained in the sample. If the sample contains a reporter molecule precursor, the first complex is also generated.

Next, as shown in FIG. 4B, the generated second complex and antigen A reporter molecule flow in the LFA test paper 20 by capillary force in the direction of the arrow AR, reaching the first detection line 23. The antigen A reporter molecule that has reached the first detection line 23 binds to the antigen A capture molecule fixed to the first detection line 23. On the other hand, the second complex does not bind to the antigen A capture molecule. As a result, the anti-antigen B labeled antibody is not captured by the first detection line 23, and no coloration occurs in the detection line on the first detection line 23. If the sample contains a reporter molecule precursor, the first complex generated in the labeling area 22 binds to the antigen A capture molecule, causing coloration of the detection line on the first detection line 23.

Next, the second complex that has passed through the first detection line 23 continues to flow on the LFA test paper 20 in the direction of the arrow AR and reaches the second detection line 24. The second complex (anti-antigen B labeled antibody of the second complex) that has reached the second detection line 24 binds to the secondary antibody of the labeled antibody fixed to the second detection line 24. The amount of anti-antigen B labeled antibody that binds to the secondary antibody of the labeled antibody increases, causing coloration of the second detection line 24. By checking the color of the second detection line 24, the inspector can confirm that the target nucleic acid has been detected in the specimen and that the test result is positive.

### Configuration of Test Kit (LFA Test Paper)

In order to accurately perform a test in accordance with the detection principle in the above-mentioned target nucleic acid detection process, it is necessary to control the amount Rp of reporter molecule precursor introduced, which is contained in the sample dropped onto the LFA test paper 20, within an allowable range. The allowable range is defined on the basis of the amount A of labeled antibodies on the LFA test paper 20 and the amount S of capture molecules on the first detection line. The inventor of the present invention found that the ratio of the amount A of labeled antibodies to the amount S of capture molecules is 1>S/A in LFA test papers in the related art, and thus the allowable range for the amount of reporter molecule precursor introduced, which is contained in the sample dropped onto the LFA test paper, is narrowed. In contrast, the LFA test paper 20 according to the present embodiment is configured such that the ratio of the amount A of labeled antibodies to the amount S of capture molecules becomes 1≤S/A, thereby widening the allowable range for the amount of reporter molecule precursor introduced. Differences between such configurations will be described below.

### Configuration in the Related Art

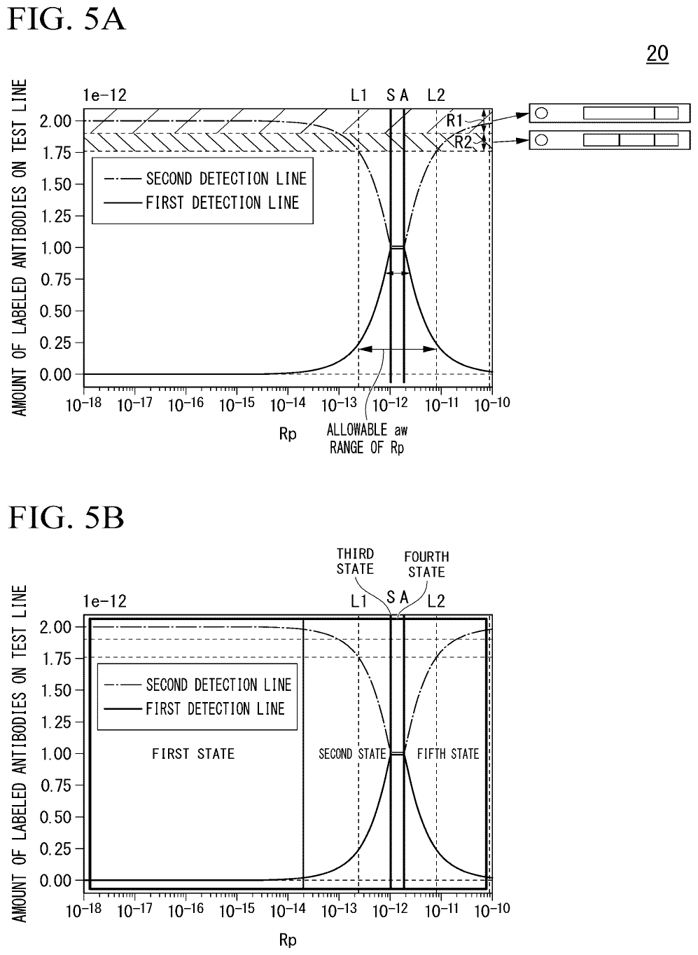

FIG. 5A and FIG. 5B are graphs showing change in the amount of labeled antibodies on each detection line with respect to the amount Rp of reporter molecule precursor introduced under negative conditions for the LFA test paper in the related art. This graph is based on a mathematical model and experimental data which will be described below. FIG. 5B shows five states (first to fifth states) on this graph.

The first state is a state in which the amount Rp of reporter molecule precursor introduced is small. In this first state, the amount of the first complex generated in the labeling area 22 is small, and thus the amount of labeled antibodies (first complex) captured on the first detection line 23 is also small. As a result, no coloration occurs on the first detection line 23. On the other hand, unbounded labeled antibody (anti-antigen B labeled antibody) flows onto the second detection line 24 and is captured. This causes coloration of the second detection line 24.

The second state is a state in which the amount of the first complex generated in the labeling area 22 increases, and the amount of the labeled antibodies (first complex) captured on the first detection line 23 also increases as the amount Rp of reporter molecule precursor introduced increases. As a result, the color of the first detection line 23 gradually becomes visible. On the other hand, the amount of unreacted labeled antibody (anti-antigen B labeled antibody) flowing onto the second detection line 24 gradually decreases. As a result, the color of the second detection line 24 gradually becomes invisible.

The third state is a state in which the amount Rp of reporter molecule precursor introduced is equal to the amount S of capture molecules on the first detection line. In this third state, the amount of labeled antibodies (first complex) captured on the first detection line 23 is the upper limit. In this state, coloration of the first detection line 23 occurs. On the other hand, the amount of unbounded labeled antibody (anti-antigen B labeled antibody) flowing onto the second detection line 24 is the lower limit. In this state, no coloration occurs on the second detection line 24.

The fourth state is a state in which the amount Rp of reporter molecule precursor introduced is between the amount S of capture molecules on the first detection line and the amount A of labeled antibodies on the LFA test paper 20. That is, in this fourth state, the amount Rp of reporter molecule precursor introduced is equal to or greater than the amount S of capture molecules and equal to or less than the amount A of labeled antibodies. During this period, the number of reporter molecule precursors bound to the labeled antibody also increases, and thus there is no effect of competition, and the amount of labeled antibodies (first complex) captured on the first detection line 23 maintains the upper peak thereof. Further, during this period, coloration of the detection line on the first detection line 23 occurs. On the other hand, the amount of unreacted labeled antibody (anti-antigen B labeled antibody) that flows onto the second detection line 24 also maintains the lower peak thereof. During this period, no coloration occurs in the detection line on the second detection line 24.

The fifth state is a state in which the amount of labeled antibodies (first complex) captured on the first detection line 23 gradually decreases as the amount Rp of reporter molecule precursor introduced increases. When the amount Rp of reporter molecule precursor introduced becomes greater than the amount A of labeled antibodies, competition occurs between the reporter molecule precursor that binds to the labeled antibodies and the reporter molecule precursor that does not bind to the labeled antibodies, and the amount of labeled antibody (first complex) captured on the first detection line 23 decreases due to the hook effect of the labeled antibodies. As a result, the color of the detection line on the first detection line 23 gradually becomes invisible. On the other hand, the amount of labeled antibody (anti-antigen B labeled antibody) that flows onto the second detection line 24 gradually increases. As a result, the color of the detection line on the second detection line 24 gradually becomes visible.

Considering the above-described five states of the LFA test paper in the related art, the allowable range for the amount Rp of reporter molecule precursor introduced can be defined as follows. That is, if the amount Rp of reporter molecule precursor introduced is excessively small, the amount of labeled antibodies on the first detection line 23 is small (first state), the amount of labeled antibodies on the first detection line 23 increases as the amount Rp of reporter molecule precursor introduced increases (second state), and if the amount Rp of reporter molecule precursor introduced is equal to or greater than the amount S of capture molecules and equal to or less than the amount A of labeled antibodies, the number of reporter molecule precursors bound to the labeled antibodies also increases, and thus no effect of competition appears (third state and fourth state). Furthermore, if the amount Rp of reporter molecule precursor introduced is greater than the amount A of labeled antibodies, competition occurs between the reporter molecule precursor bound to the labeled antibody and the reporter molecule precursor not bound to the labeled antibody, and the amount of labeled antibodies (first complex) captured on the first detection line 23 due to the hook effect of the labeled antibodies decreases (fifth state). Therefore, the amount of labeled antibodies on the first detection line 23 is the largest and the amount of labeled antibodies on the second detection line 24 is the smallest under the condition that the amount Rp of reporter molecule precursor introduced is in the range of the amount S of capture molecules on the first detection line and the amount A of labeled antibodies. A range in which coloration of the first detection line 23 occurs and no coloration occurs in the second detection line 24 (the range between L1 and L2 shown in FIG. 5A) having the range from the amount S of capture molecules on the first detection line to the amount A of labeled antibodies at the center, is the allowable range for the amount Rp of reporter molecule precursor introduced. That is, the design range of the amount Rp of reporter molecule precursor introduced is determined by the ratio (difference) between the amount A of labeled antibodies A and the amount S of capture molecules.

As described above, the width of the peak where the amount of labeled antibodies on the first detection line 23 is the largest and the amount of labeled antibodies on the second detection line 24 is the smallest determines the width to the foot of the graph and affects the allowable range for the amount Rp of reporter molecule precursor introduced. In the configuration in the related art shown in FIG. 5A, the amount S of capture molecules is 1.0×10⁻¹² mol, the amount A of labeled antibodies is 2.0×10⁻¹² mol, and the allowable range for the amount Rp of reporter molecule precursor introduced is narrowed from 2.4×10⁻¹³ mol/LFA to 8×10⁻¹² mol/LFA.

### Design Method of Present Embodiment

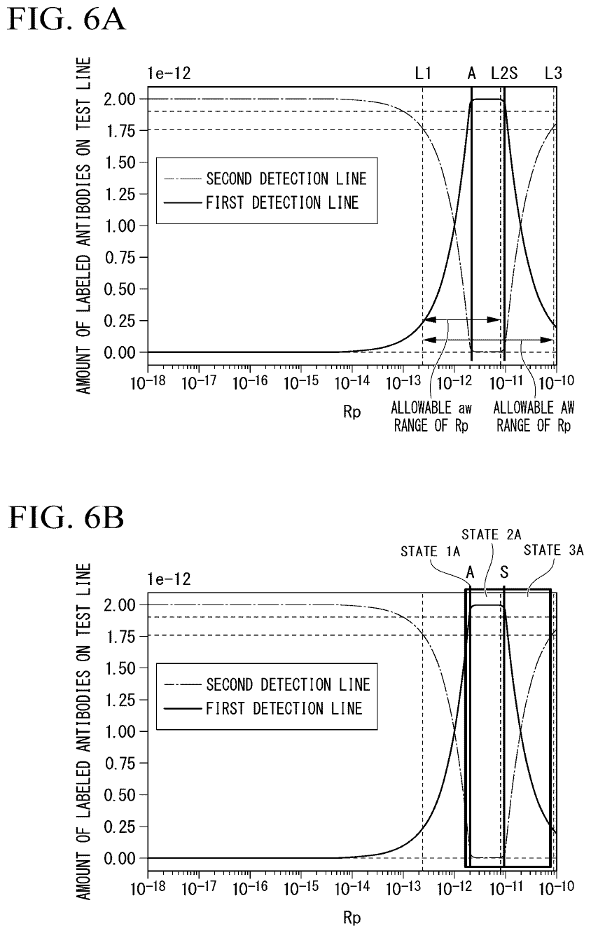

FIG. 6A and FIG. 6B are graphs showing change in the amount of labeled antibodies on each detection line with respect to the amount Rp of reporter molecule precursor introduced under negative conditions for the LFA test paper according to the embodiment. This graph is based on a mathematical model and experimental data which will be described below. FIG. 6B shows three states (states 1A to 3A) on this graph.

State 1A is a state in which the amount Rp of reporter molecule precursor introduced is equal to the amount A of labeled antibodies. In this state 1A, the amount of labeled antibodies (first complex) captured on the first detection line 23 is the upper limit. In this state, coloration of the detection line on the first detection line 23 occurs. On the other hand, the amount of unreacted labeled antibody (anti-antigen B labeled antibody) that flows onto the second detection line 24 is the lower limit. In this state, no coloration occurs in the detection line on the second detection line 24.

State 2A is a state in which the amount Rp of reporter molecule precursor introduced is between the amount A of labeled antibodies and the amount S of capture molecules on the first detection line 23. That is, in this state 2A, the amount Rp of reporter molecule precursor introduced is equal to or greater than the amount A of labeled antibodies and less than the amount S of capture molecules. The hook effect does not occur until the amount Rp of reporter molecule precursor introduced becomes equal to or greater than the amount S of capture molecules. Therefore, the amount of labeled antibodies (first complex) captured on the first detection line 23 maintains the upper limit peak and does not change. During this period, coloration of the detection line on the first detection line 23 occurs. On the other hand, the amount of unreacted labeled antibody (anti-antigen B labeled antibody) flowing onto the second detection line 24 also maintains the lower limit peak. During this period, no coloration occurs in the detection line on the second detection line 24.

State 3A is a state in which the amount Rp of reporter molecule precursor introduced is equal to or greater than the amount S of capture molecules. In this state 3A, as the amount Rp of reporter molecule precursor introduced increases, the amount of labeled antibodies (first complex) captured on the first detection line 23 gradually decreases. This is because when the amount Rp of reporter molecule precursor introduced becomes greater than the amount S of capture molecules, competition occurs on the first detection line 23 due to the hook effect, and the amount of captured labeled antibodies (first complex) decreases. As a result, the color of the detection line on the first detection line 23 gradually becomes invisible. On the other hand, the amount of labeled antibodies (anti-antigen B labeled antibody) on the second detection line 24 gradually increases. As a result, the detection line on the second detection line 24 gradually becomes visible.

Considering the three states of the LFA test paper 20 according to the present embodiment as described above, the allowable range for the amount Rp of reporter molecule precursor introduced can be theoretically defined as follows. That is, as in the configuration in the related art, even if the amount S of capture molecules is reduced under the condition that the amount A of labeled antibodies is greater than the amount S of capture molecules (S<A), the amount of leakage of xx at the peak will increase, and in reality, the allowable range for the amount Rp of reporter molecule precursor introduced will not be widened. Similarly, even if the amount A of labeled antibodies is increased, the allowable range for the amount Rp of reporter molecule precursor introduced will not be widened. Even if there is a difference between amount S of capture molecules and the amount A of labeled antibodies under the condition that the amount A of labeled antibodies is greater than the amount S of capture molecules (S<A), the height of the upper limit (peak) of the amount of labeled antibodies captured on the first detection line 23 will change, and the allowable range for the amount Rp of reporter molecule precursor introduced will not be widened.

On the other hand, if the amount A of labeled antibodies is reduced drastically under the condition that the amount S of capture molecules is greater than the amount A of labeled antibodies (S>A), the color on the second detection line 24 will not be observed in the positive case. For this reason, in the present embodiment, under conditions that the amount S of capture molecules is equal to or greater than the amount A of labeled antibodies (S≥A) and the amount A of labeled antibodies is equal to or greater than a predetermined amount (A≥10⁻¹² mol), a range in which coloration of the first detection line 23 occurs and no coloration occurs in the second detection line 24 (the range between L1 and L3 shown in FIG. 6A) having the range from the amount A of labeled antibodies to the amount S of capture molecules on the first detection line at the center becomes the allowable range for the amount Rp of reporter molecule precursor introduced. Within this allowable range, the width of the above-mentioned state 2A (state in which the amount Rp of reporter molecule precursor introduced is equal to or greater than the amount A of labeled antibodies and less than the amount S of capture molecules) is the optimal allowable range. By designing the amount S of capture molecules to be equal to or greater than the amount A of labeled antibodies (S≥A), the width of the peak where the amount of labeled antibodies on the first detection line 23 is the upper limit becomes wider than that in the related art, and the allowable range can be widened.

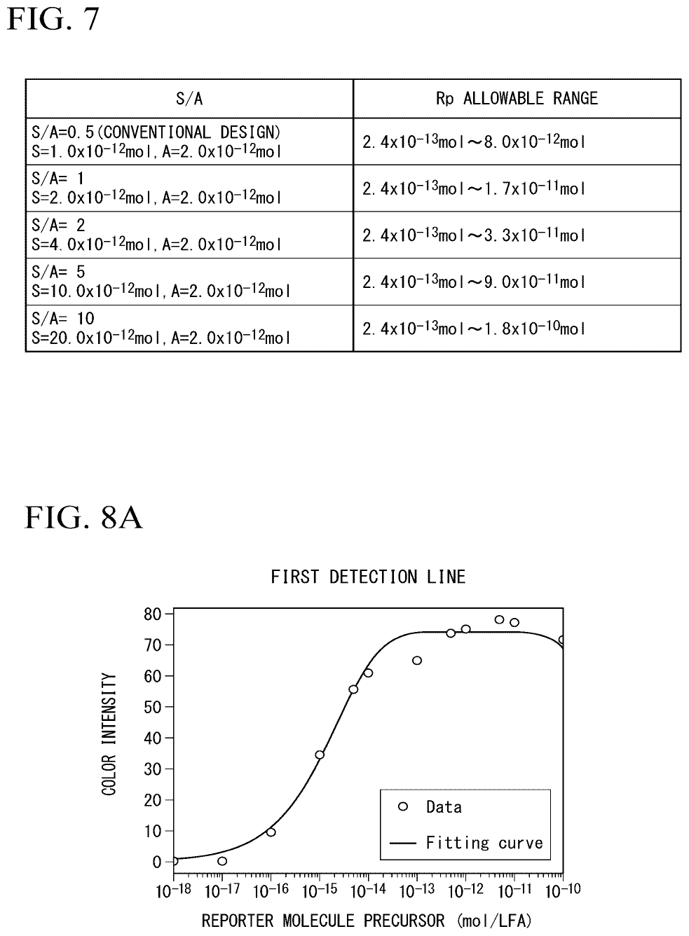

FIG. 7 is a diagram showing a relationship between the allowable range for the amount Rp of reporter molecule precursor introduced (Rp allowable range) and the ratio S/A of the amount S of capture molecules to the amount A of labeled antibodies on the first detection line according to the embodiment when the ratio S/A is changed. These values are calculated using a mathematical model which will be described below. It was confirmed that when the amount S of capture molecules on the first detection line 23 is equal to or greater than the amount A of labeled antibodies (S≥A), the width of the peak where the amount of labeled antibodies on the first detection line 23 is the upper limit becomes wider than that in the related art, and the allowable range is widened. This enables a more robust test to be performed against fluctuations in the amount Rp of reporter molecule precursor introduced.

The test kit 1 is an example of a “test kit.” The LFA test paper 20 is an example of an “LFA test paper.” The test kit 1 detects a target nucleic acid sequence in a specimen. The test kit 1 includes the LFA test paper 20 for testing a reaction solution obtained by adding a specimen to a reaction solution containing a Cas enzyme and a reporter molecule precursor and subjecting the reaction solution to a CRISPR-Cas reaction. The LFA test paper 20 includes the labeling area 22 containing a labeled antibody, the first detection line 23 to which capture molecules that capture the reporter molecule precursor are fixed, and a second detection line 24 to which capture antibodies that capture the labeled antibodies are fixed. The amount A of labeled antibodies and the amount S of capture molecules on the LFA test paper 20 satisfy the conditions of A≥10⁻¹² mol and 1≤S/A. The amount Rp of reporter molecule precursor introduced into the LFA test paper 20 satisfies the condition of A≤Rp<S.

Preferably, the amount A of labeled antibodies and the amount S of capture molecules may further satisfy the condition of 2≤S/A. More preferably, the amount A of labeled antibodies and the amount S of capture molecules may further satisfy the condition of 5≤S/A. More preferably, the amount A of labeled antibodies and the amount S of capture molecules may further satisfy the condition of 10≤S/A.

Preferably, the amount A of labeled antibodies and the amount S of capture molecules may further satisfy the conditions of A≥2×10⁻¹² mol and 1≤S/A. More preferably, the amount A of labeled antibodies and the amount S of capture molecules may further satisfy the conditions of A≥2×10⁻¹² mol and 2≤S/A. More preferably, the amount A of labeled antibodies and the amount S of capture molecules may further satisfy the conditions of A≥2×10⁻¹² mol and 5≤S/A. More preferably, the amount A of labeled antibodies and the amount S of capture molecules may further satisfy the conditions of A≥2×10⁻¹² mol and 10≤S/A.

### Mathematical Model

Hereinafter, the mathematical model used to determine the configuration of the test kit according to the present embodiment will be described. This mathematical model is based on the following two settings.

### Setting 1: Complex Formation

All molecules meet immediately after mixing with binding molecules, and molecules introduced into the system are given the opportunity to bind (equilibrium is reached immediately, no inactivation occurs). Furthermore, a complex is formed at a binding rate that depends on the amount of molecules and the strength of affinity.

### Setting 2: Complex Binding (Related to the Hook Effect)

A complex is formed at a binding rate that depends on the amount of molecules and the strength of affinity. This binding rate between target molecules does not change depending on the presence or absence of binding of another molecule.

### Initial Conditions

The initial conditions of the mathematical model are as follows.

CRISPR-Cas reaction process:
Amount of template [N₀]≥0
Amount of enzyme [E₀]≥0
Amount of reporter molecule precursor [Rp₀]>0

LFA test paper:
Amount of labeled antibody [A₀]>0
Amount of capture molecular of reporter molecule precursor [S₀]>0
Amount of capture antibody of labeled antibody [G₀]>0

### (1) Mathematical Model (CRISPR-Cas Reaction)

### Binding of Template DNA and Cas Enzyme

Binding of template DNA and Cas enzyme is further assumed as follows. That is, template DNA and Cas enzyme bind through the binding process of assumption 1 above. All Cas enzymes bound to the template DNA become active enzymes. The amount of reporter molecule precursor cleaved by a unit amount of active enzyme per unit time is defined as enzyme activity A. The enzyme activity A is a constant determined only by the type of enzyme and does not depend on the amount of reporter molecule precursor or enzyme.

The total amount of template DNA in the reaction system is denoted by [N₀], and the total amount of Cas enzyme is denoted by [E₀]. Here, if the equilibrium dissociation constant of the bond between these two molecules is denoted by K_{NE}, the amount of active enzyme [NE] can be calculated using the following formulas (1) to (5).

$$\begin{matrix}N + E & \begin{matrix}{K}_{on} \\ ⟶ \\ ⟵ \\ {K}_{off}\end{matrix} & NE\end{matrix} \qquad Formula ( 1 )$$

$$d [ NE ] / dt = {K}_{on} [ N ] [ E ] - {K}_{off} [ KE ] = 0 \qquad Formula ( 2 )$$

Here, because [N]=[N₀]−[NE], and [E]=[E₀]−[NE], the above formula (2) can be rewritten as the following formula (3).

$$d [ NE ] / dt = {K}_{on} ( [ {N}_{0} ] - [ NE ] ) ( [ {E}_{0} ] - [ NE ] ) - {K}_{off} [ NE ] = 0 \qquad Formula ( 3 )$$

By using K_{NE}=K_{off}/Kₒₙ, the above formula (3) can be rewritten as the following formula (4).

$${[ NE ]}^{2} - [ NE ] ( [ {N}_{0} ] + [ {E}_{0} ] + {K}_{NE} ) + [ {N}_{0} ] [ {E}_{0} ] = 0 \qquad Formula ( 4 )$$

The following formula (5) can be obtained by solving the quadratic equation of the above formula (4).

$$[ NE ] = \frac{\begin{matrix}( [ {N}_{0} ] [ {E}_{0} ] KNE ) - \\ \sqrt{{( [ {N}_{0} ] + [ {E}_{0} ] + KNE )}^{2} - 4 [ {N}_{0} ] [ {E}_{0} ]}\end{matrix}}{2} \qquad Formula ( 5 )$$

### Cleavage of Reporter Molecule Precursor by Active Enzyme

The enzyme activity is denoted by A, the reaction time is denoted by T, and the amount of active enzyme is denoted by [NE]. The amount of reporter molecule precursor to be cleaved is represented by AT [NE]. When [Rp₀]>AT [NE], the following is obtained.

$$[ Rp ] = [ {Rp}_{0} ] - AT [ NE ]$$

$$[ R 1 ] = AT [ NE ]$$

$$[ R 2 ] = AT [ NE ]$$

Further, when [Rp₀]≤AT[NE], all initial reporter molecule precursors are cleaved, and the following is obtained.

$$[ Rp ] = 0$$

$$[ R 1 ] = [ {Rp}_{0} ]$$

$$[ R 2 ] = [ {Rp}_{0} ]$$

In the cleavage reaction of the reporter molecule precursor in CRISPR-Cas reaction, the enzyme reaction rate equation may be used. For example, the total quasi-steady-state approximation (tQSSA), which is more accurate than the Michaelis-Menten equation, or the like may be used.

### (2) Mathematical Model (LFA)

### Binding of Labeled Antibody to Reporter Molecule Precursor or First Reporter Molecule

It is assumed that all of introduced CRISPR-Cas reaction products flow to the labeling area 22 (conjugation pad), and the labeled antibody in the labeling area 22 has the ability to bind to all of reporter molecule precursors or the first reporter molecule (e.g., antigen B reporter molecule). The amount of reporter molecule precursor is denoted by [Rp], the first reporter molecular is denoted by [R1], the amount of labeled antibody is denoted by [A], the total amount of labeled antibody-bound molecules is denoted by [RA], the amount of labeled antibody-bound reporter molecule precursor is denoted by [RpA], and the labeled antibody-bound first reporter molecular is denoted by [R1A].

In this reaction system, the total amount of reporter molecule precursor and first reporter molecule is denoted by [R₀], and the total amount of labeled antibodies is denoted by [A₀]. Here, if the equilibrium dissociation constant of the bond between these two molecules is K_{RA}, the total amount (RA) of labeled antibody-bound molecules is represented by the following formula (6).

$$[ RA ] = \frac{\begin{matrix}( [ {Rp}_{0} ] + [ {A}_{0} ] + KRA ) - \\ \sqrt{{( [ {Rp}_{0} ] + [ {A}_{0} ] + KRA )}^{2} - 4 [ {Rp}_{0} ] [ {A}_{0} ]}\end{matrix}}{2} \qquad Formula ( 6 )$$

The amount of labeled antibody-bound reporter molecule precursor, [RpA], the labeled antibody-bound first reporter molecular [R1A], and the amount of reporter molecule precursor not bound to the labeled antibody, [R], are represented as follows using [RA].

$$[ RpA ] = [ Rp ] [ RA ] / [ {Rp}_{0} ]$$

$$[ R 1 A ] = [ R 1 ] [ RA ] / [ {Rp}_{0} ]$$

$$[ R ] = [ Rp ] - [ RpA ]$$

### Binding at First Detection Line

Assuming the binding at the first detection line 23 as follows. All molecules flow into the first detection line 23. All capture molecules at the first detection line 23 have the ability to bind to the reporter molecule precursor or the second reporter molecule (e.g., antigen A reporter molecule). Binding between the capture molecule and the reporter molecule precursor occurs regardless of whether or not the labeled antibody is bound. Molecules not captured at the first detection line 23 flow to the second detection line 24.

An unbound free capture molecule is denoted by [S], the total amount of molecules bound to the capture molecule is denoted by [RS], the amount of labeled antibody-bound reporter molecule precursor bound to the capture molecule is denoted by [RpAS], the amount of labeled antibody-unbound reporter molecule precursor bound to the capture molecule is denoted by [RpS], and the capture module-bound second reporter molecular is denoted by [R2S].

The total amount of the second reporter molecule and reporter molecule precursor in this reaction system is denoted by [Rp₀], and the total amount of capture molecules is denoted by [S₀]. If the equilibrium dissociation constant of the bond between these two molecules is K_{RS}, the total amount of reporter bound to the capture molecule, [RS], is represented by the following formula (7).

$$[ RS ] = \frac{\begin{matrix}( [ {Rp}_{0} ] + [ {S}_{0} ] + KRS ) - \\ \sqrt{{( [ {Rp}_{0} ] + [ {S}_{0} ] + KRS )}^{2} - 4 [ {Rp}_{0} ] [ {S}_{0} ]}\end{matrix}}{2} \qquad Formula ( 7 )$$

The amount of labeled antibody-bound reporter molecule precursor bound to the capture molecule, [RpAS], the amount of labeled antibody-unbound reporter molecule precursor bound to the capture molecule, [RpS], and the capture molecule-bound second reporter molecular [R2S] are represented as follows.

$$[ RpAS ] = [ RpA ] [ RS ] / [ {Rp}_{0} ]$$

$$[ RpS ] = [ R ] [ RpS ] / [ {Rp}_{0} ]$$

$$[ R 2 S ] = [ R 2 ] [ RS ] / [ {Rp}_{0} ]$$

$$[ S ] = [ {S}_{0} ] - [ RS ]$$

### Binding at Second Detection Line

Binding at the second detection line 24 is assumed as follows. All molecules that do not bind at the first detection line 23 flow to the second detection line 24. All capture antibodies on the second detection line 24 have the ability to bind to labeled antibodies. Binding between the capture antibodies and the labeled antibodies occurs regardless of whether or not binding to the reporter molecule precursor or the first reporter molecule occurs. Molecules that are not captured at the second detection line 24 flow to the absorbent pad 25.

An unbound free capture antibody is denoted by [G], and the total amount of AuNP antibodies bound to the capture antibody is denoted by [AG]. The total amount of labeled antibodies in this reaction system is denoted by [Aₜ], and the total amount of capture antibodies is denoted by [G₀]. If the equilibrium dissociation constant of the bond between these two molecules is K_{AG}, the total amount of labeled antibodies bound to the capture antibody, [AG], is represented by the following formula (8).

$$[ AG ] = \frac{\begin{matrix}( [ {A}_{t} ] + [ {G}_{0} ] + KAG ) - \\ \sqrt{{( [ {A}_{t} ] + [ {G}_{0} ] + KAG )}^{2} - 4 [ At ] [ {G}_{0} ]}\end{matrix}}{2} \qquad Formula ( 8 )$$

Here, the amount of labeled antibodies flowing from the first detection line 23 to the second detection line 24 is [A₀]−[RpAS], and thus [At]=[A₀]−[RpAS].

### (3) Mathematical Model (Color Intensity Conversion)

The color intensity of each detection line can be represented from the concentration of labeled antibodies on each detection line using the 5PL model used in immunoassays such as ELISA. The 5PL model is represented by the following formula (9).

$$y = d + \frac{a - d}{{[ a + {( \frac{x}{c} )}^{b} ]}^{g}} \qquad Formula ( 9 )$$

In the above formula (9), a is the theoretical response at zero concentration, b is the slope factor, c is the intermediate concentration (inflection point), d is the theoretical response at infinite concentration, and g is the asymmetry factor.

### Setting Parameters of Mathematical Model

Parameters set in the mathematical model include the amount of labeled antibodies, the capture molecular on the first detection line, and parameters on each detection line of the color intensity conversion formula 5PL model. The implementation procedure is as follows: first, the color intensity of each detection line is measured when the amount of reporter molecule precursor has been changed under negative conditions, and the mathematical model formula is fitted to the measured values and the data of the amount of reporter molecule precursor, and each of the above parameters is obtained.

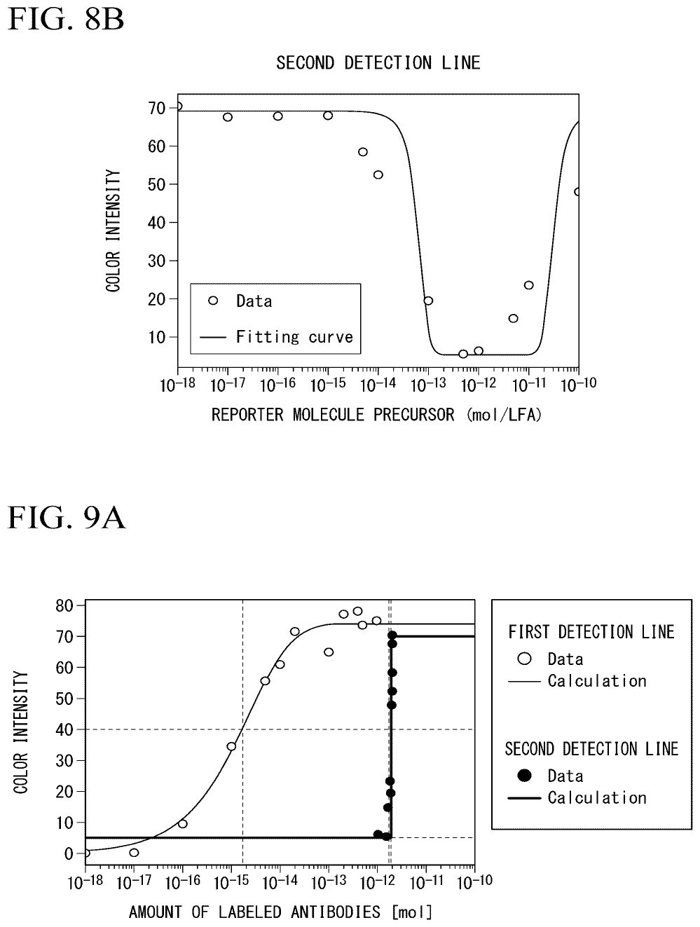

FIG. 8A shows change in color intensity with respect to the amount of reporter molecule precursor in the first detection line 23 according to the embodiment. As a result of fitting the mathematical model formula to the data of the amount of reporter molecule precursor shown in FIG. 8A, the following parameters are obtained: labeled antibody amount: 2×10⁻¹² mol, capture molecular: 1×10⁻¹² mol, 5PL model parameters: a=0.1, b=5.8×10⁻¹, c=4.3×10⁻¹³, d=74, g=5.4. As shown in FIG. 8A, the mathematical model formula in which these parameters are set well represents the relationship between the amount of reporter molecule precursor and the color intensity of the first detection line 23. The dissociation constant was assumed to be sufficiently smaller than the amount of introduced substances.

FIG. 8B shows change in color intensity with respect to the amount of reporter molecule precursor in the second detection line 24 according to the embodiment. As a result of fitting the mathematical model formula to the data of the amount of reporter molecule precursor shown in FIG. 8B, the following parameters were obtained: 5PL model parameters: a=5.26.1, b=100, c=1.9×10^{-11,} d=70, g=1.67. As shown in FIG. 8B, the mathematical model formula in which these parameters are set well represents the relationship between the amount of reporter molecule precursor and the color intensity of the second detection line 24. The amount of capture antibodies was set to 2×10⁻¹² mol, which is equal to the amount of labeled antibodies.

### Verification

The following verification was performed using the mathematical model in which the parameters are set as above. Specifically, it was verified whether a design of A=2×10⁻¹² mol, A≤Rp<S, 1<S/A could suppress a decrease in specificity with respect to variation in the amount Rp of reporter molecule precursor introduced, compared to the design in the related art.

### Verification 1

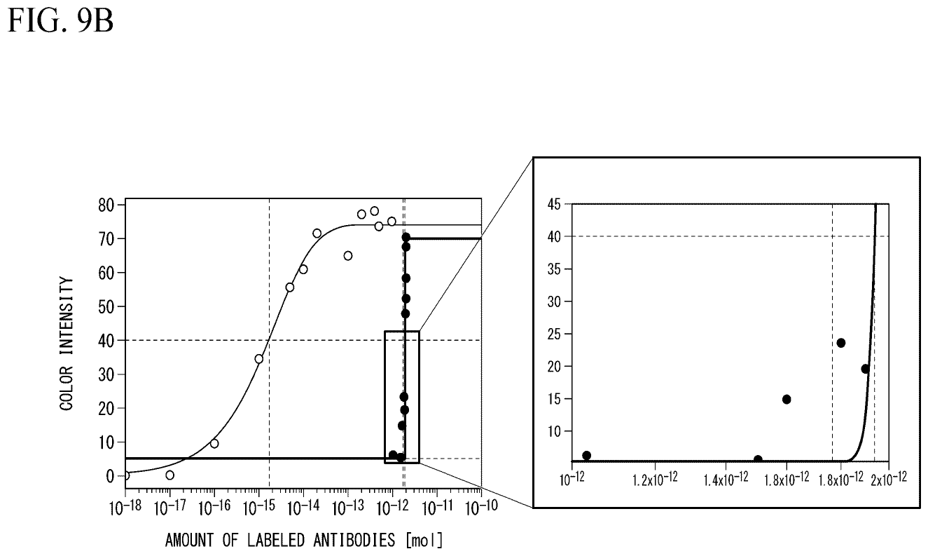

A graph showing the amount of labeled antibodies and the color intensity of each line was plotted from measured values and the mathematical model. A threshold value for determining the color state of each line and the amount of labeled antibodies at that threshold value were determined from the graph. In addition, the amount of labeled antibodies required for the second detection line to show color in a positive case was determined from the graph. FIG. 9A is a graph showing the relationship between the amount of labeled antibodies and the color intensity according to the embodiment. In this verification 1, a threshold value for the color intensity at which a detection line is determined to be colored was “40” and a threshold value for the color intensity at which the detection line is determined to be not colored was “5.2.” Furthermore, the amount of labeled antibodies in the first detection line 23 was calculated to be 1.7×10⁻¹⁵ mol, and the amount of labeled antibodies in the second detection line 24 was calculated to be 2.0×10⁻¹² mol when the color intensity was 40. The amount of labeled antibodies in the second detection line 24 was calculated to be 1.75×10⁻¹² mol when the color intensity was 5.2.

FIG. 9B is an enlarged view of a part of the graph shown in FIG. 9A. It was found that the labeled antibody showed coloration on the order of 10⁻¹² mol, and that coloration was sufficiently observed if the amount was 2×10⁻¹² mol or more. The amount of labeled antibodies and the capture molecular shown in the present are amounts calculated as parameters by fitting the aforementioned mathematical model with actual measured values.

### Verification 2

Random numbers were generated for the amount Rp of reporter molecule precursor introduced having a certain variation, specificity was calculated when S/A was changed based on the mathematical model and the threshold values determined in the above verification 1, and changes in specificity with respect to the variation in the amount Rp of reporter molecule precursor introduced in the design (S/A<1) in the related art and the design of the present embodiment (S/A≥1) was compared. As a procedure, random numbers for the amount Rp of reporter molecule precursor introduced having a certain variation were generated (log normal distribution). These became random numbers for samples under negative conditions. Next, the random numbers were substituted into the mathematical model to calculate the amount of labeled antibodies on each detection line, and when the amount of labeled antibodies on each detection line was compared with the threshold value, the number of samples that were determined to be negative is counted and the specificity was calculated.

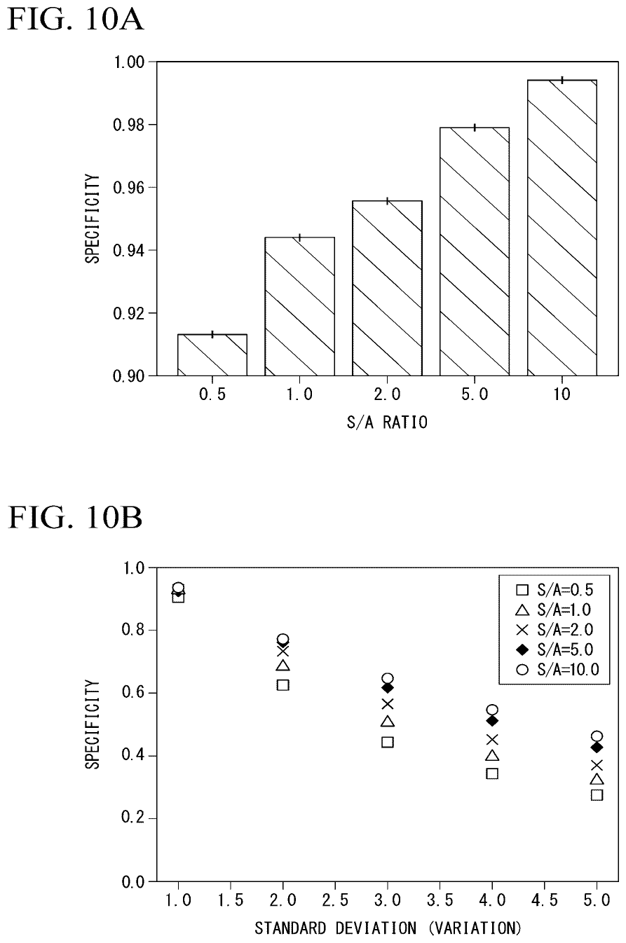

FIG. 10A is a graph showing change in specificity when the ratio S/A of the amount S of capture molecules to the amount A of labeled antibodies on the first detection line according to the embodiment has been changed. FIG. 10A shows the result of calculating the specificity by simulation using the mathematical model when the amount Rp of reporter molecule precursor introduced is varied. The specificity indicates a proportion of negative results that were determined to be negative. As shown in FIG. 10A, it was confirmed that the specificity increases when the ratio S/A of the amount S of capture molecules to the amount A of labeled antibodies is increased.

FIG. 10B is a graph showing change in specificity with respect to variation (standard deviation) in the amount Rp of reporter molecule precursor introduced according to the embodiment. FIG. 10B shows the results of calculating the specificity by simulation using the mathematical model when the amount Rp of reporter molecule precursor introduced is varied. As shown in FIG. 10B, it was confirmed that the decrease in specificity is small and the robustness of the test is high when the ratio S/A of the amount S of capture molecules to the amount A of labeled antibodies is increased.

### Verification 3

Changes in sensitivity with respect to variation in the amount Rp of reporter molecule precursor introduced were compared. Sensitivity refers to a proportion of positive results that could be determined to be positive. As a procedure, random numbers for the amount Rp of reporter molecule precursor introduced when 99.999% of reporter molecule precursor having a certain variation was converted to reporter molecules (when the cleavage ratio of the reporter molecule precursor was 99.999%) were generated (log normal distribution). These became random numbers for samples under positive conditions. Next, the random numbers were substituted into the mathematical model, the amount of labeled antibodies on each detection line was calculated, and the amount of labeled antibodies on each detection line was compared with the threshold value, the number of samples determined to be positive was counted, and the sensitivity was calculated.

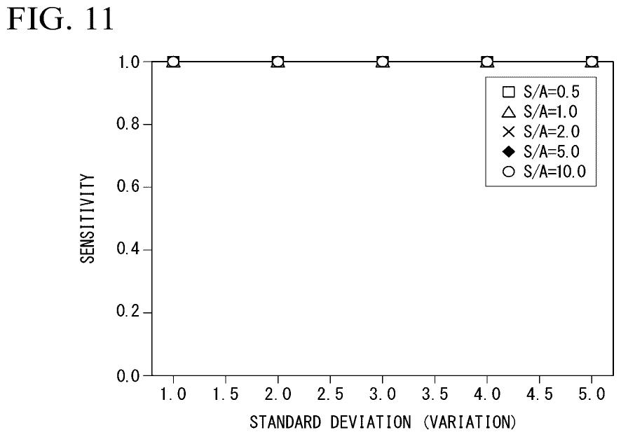

FIG. 11 is a graph showing change in sensitivity with respect to variation (standard deviation) in the amount Rp of reporter molecule precursor introduced according to the embodiment. FIG. 11 shows results of calculating the sensitivity by simulation using the mathematical model when the amount Rp of reporter molecule precursor introduced is varied. As shown in FIG. 11, it was confirmed that a variation in the amount Rp of reporter molecule precursor introduced does not affect the sensitivity and that the sensitivity does not change even if the ratio S/A of the amount S of capture molecules to the amount A of labeled antibodies is changed.

The test of the present embodiment may be performed while improving the test sensitivity by combining CRISPR-Cas LFA with a nucleic acid amplification reaction. After amplifying a target nucleic acid sequence in a specimen by the nucleic acid amplification reaction, the amplified products are subjected to a CRISPR-Cas reaction to perform a LFA test. For the nucleic acid amplification reaction, for example, an isothermal nucleic acid amplification method such as PCR, LAMP, or RPA may be used. As long as the target nucleic acid sequence can be amplified according to the test environment, there is no limitation on the method, whether it is a PCR method that requires temperature control or LAMP, RPA, etc. that do not require temperature control.

According to the embodiment described above, by widening the allowable range of the amount of reporter molecule precursor introduced, it is possible to perform a test that is robust against fluctuations in the amount of introduced reporter molecule precursors.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A test kit for detecting a target nucleic acid sequence in a specimen, the test kit comprising a lateral flow assay (LFA) test paper for testing a reaction solution obtained by adding the specimen to a reaction solution containing a Cas enzyme and a reporter molecule precursor and subjecting the reaction solution to a CRISPR-Cas reaction,
wherein the LFA test paper comprises a labeling area containing labeled antibodies, a first detection line to which capture molecules for capturing the reporter molecule precursor are fixed, and a second detection line to which capture antibodies for capturing the labeled antibodies are fixed,
wherein the amount A of the labeled antibodies and the amount S of the capture molecules on the LFA test paper satisfy conditions of A≥10⁻¹² mol and 1≤S/A.

2. The test kit according to claim 1, wherein the amount Rp of the reporter molecule precursor introduced into the LFA test paper satisfies a condition of A≤Rp<S.

3. The test kit according to claim 1, wherein the amount A of the labeled antibodies and the amount S of the capture molecules further satisfy a condition of 2≤S/A.

4. The test kit according to claim 1, wherein the amount A of the labeled antibodies and the amount S of the capture molecules further satisfy a condition of 5≤S/A.

5. The test kit according to claim 1, wherein the amount A of the labeled antibodies and the amount S of the capture molecules further satisfy a conditions of 10≤S/A.

6. The test kit according to claim 1, wherein the amount A of the labeled antibodies and the amount S of the capture molecules further satisfy conditions of A≥2×10⁻¹² mol and 1≤S/A.

7. The test kit according to claim 1, wherein the amount A of the labeled antibodies and the amount S of the capture molecules further satisfy conditions of A≥2×10⁻¹² mol and 2≤S/A.

8. The test kit according to claim 1, wherein the amount A of the labeled antibodies and the amount S of the capture molecules further satisfy conditions of A≥2×10⁻¹² mol and 5≤S/A.

9. The test kit according to claim 1, wherein the amount A of the labeled antibodies and the amount S of the capture molecules further satisfy conditions of A≥2×10⁻¹² mol and 10≤S/A.

10. The test kit according to claim 1, wherein the LFA test paper is configured to test a reaction solution obtained by subjecting amplified products generated by amplifying a target nucleic acid sequence in the specimen by a nucleic acid amplification reaction to a CRISPR-Cas reaction.

11. A method of designing a test kit for detecting a target nucleic acid sequence in a specimen,
wherein the test kit comprises a lateral flow assay (LFA) test paper for testing a reaction solution obtained by adding the specimen to a reaction solution containing a Cas enzyme and a reporter molecule precursor and subjecting the reaction solution to a CRISPR-Cas reaction,
wherein the LFA test paper includes a labeling area containing labeled antibodies, a first detection line to which capture molecules for capturing the reporter molecule precursor are fixed, and a second detection line to which capture antibodies for capturing the labeled antibodies are fixed,
the method comprising setting the amount A of the labeled antibodies and the amount S of the capture molecules on the LFA test paper such that the amount A of the labeled antibodies and the amount S of the capture molecules on the LFA test paper satisfy conditions of A≥10⁻¹² mol and 1≤S/A.
